(19) United States
(12) Patent Application Publication
(10) Pub. No.: US 2016/0272665 A1
(43) Pub. Date: Sep. 22, 2016

(54) PRODUCTION OF RENEWABLE FINE CHEMICALS AND LIQUID FUELS

(71) Applicant: PURDUE RESEARCH FOUNDATION, West Lafayette, IN (US)

(72) Inventors: Nicholas William Delgass, West Lafayette, IN (US); Rakesh Agrawal, West Lafayette, IN (US); Fabio Henrique Ribeiro, West Lafayette, IN (US); Sara Lynn Yohe, West Lafayette, IN (US); John Charles Degenstein, West Lafayette, IN (US)

(73) Assignee: PURDUE RESEARCH FOUNDATION, West Lafayette, IN (US)

(21) Appl. No.: 15/032,529

(22) PCT Filed: Oct. 27, 2014
(86) PCT No.: PCT/US2014/062475

§ 371 Date: Apr. 27, 2016

Related U.S. Application Data
(60) Provisional application No. 61/896,119, filed on Oct. 27, 2013.

(51) Int. Cl. C07G 1/00; C07C 41/18; C07C 1/24; C07C 5/367; C07C 4/02; C08H 8/00; C07C 2/56; C07C 45/00; C07C 5/327; C12P 7/06; C10L 1/04; C10L 1/02; C13K 1/02; C12P 7/16; C07C 37/50

(52) U.S. Cl. CPC C07G 1/00; C07C 37/50; C07C 41/18; C07C 1/24; C07C 5/367; C07C 4/02; C08H 8/00; C07C 2/56; C07C 45/00; C07C 5/327; C12P 7/06; C10L 1/04; C10L 1/02; C13K 1/02; C12P 7/16; C07C 2101/14; C07C 2101/16; C10L 2200/0469

(57) ABSTRACT

Described herein is a process for conversion and upgrading of biomass to products. The process involves converting the biomass to primary lignin-derivatives and primary cellulose/hemicellulose derivatives, catalytically treating the primary lignin-derivatives to produce secondary lignin-derived products, and treating the primary cellulose/hemicellulose- derivatives to produce secondary cellulose/hemicellulose-derived products.

The present non-provisional patent application is related to and claims the priority benefit of U.S. Provisional Patent Application Ser. No. 61/896,119, filed Oct. 27, 2013, the contents of which is hereby incorporated by reference in its entirety into the present disclosure.

### STATEMENT OF GOVERNMENT SUPPORT

This invention was made with government support under DGE-1333468 awarded by the National Science foundation, DE-FG36-08G018087 awarded by the U.S. Department of Energy, DGE-0938033 awarded by the National Science Foundation, and DE-SC0000997 awarded by the U.S. Department of Energy. The government has certain rights in the invention.

### TECHNICAL FIELD

The present disclosure generally relates to biorefineries, and in particular to lignin processing methods for the production of renewable fine chemicals and liquid fuels.

### BACKGROUND

The interest and demand for renewable alternatives to petroleum-derived fuels and chemicals has grown substantially in the last decade. Biomass is a promising feedstock for sustainable commodities as it presents the only source of renewable carbon. As the technology for production of renewable fuels and chemicals from biomass has progressed, biorefinery concepts have been developed and some (based on fermentation and liquid phase upgrading) have been commercially implemented. As the U.S. transitions to a new energy landscape, any renewable platform must be able to provide both liquid fuels and commodity chemicals on a large scale. For example, in 2012 the U.S. consumed ˜4.75 billion barrels per year and greater than 50 Mton of liquid fuels and organic commodity chemicals, respectively (including ˜15 Mton/yr of propylene and ˜10 Mton/yr of benzene). Current commercial biorefineries and biorefinery concepts however focus mainly on the cellulose/hemicellulose (or sugar) fraction of biomass for the production of fuels and chemicals and neglect the lignin fraction (which is often used for process heat and power), lowering the energy and carbon utilization of the biomass feedstock. Lignin, composed of ether linked propylphenolic units and comprising up to 40% of the energy and, on average, 25 wt % of biomass, is a valuable component of lignocellulosic biomass. It represents an opportunity for increased product yield and production of high-value products, specifically renewable aromatic commodity chemicals (such as benzene, toluene, xylene (BTX), styrene, and cumene) which would utilize the natural aromatic backbone present in lignin.

Lignin has been utilized in the past to produce high value products such as high octane aromatic fuels or chemicals such as BTX and other aromatics. However, actual reaction schemes to effectively convert lignin with high yield to useful end products remains a significant challenge, and catalyst development for selective lignin conversion was identified in PNNL's 2007 report on biorefinery lignin as a core area of future R&D needed for lignin implementation in future biorefineries. Current processing techniques for lignin have drawbacks that limit their commercial and scientific feasibility. Biorefineries based on a sugar platform coproduce lignin as sulfite, kraft, and soda lignin from the pulp and paper industry, or as a lignin byproduct from lignocellulosic bioethanol production. However, the lignin byproducts from processes that focus on the carbohydrate fraction are either still recalcitrant polymers or impure and complex mixtures of compounds, which greatly increases the difficulty of effective upgrading. Combustion and gasification processes focus on conversion all of the lignocellulosic biomass, however they involve completely breaking down the biomass structure into small molecules, which results in a loss of the valuable natural aromatic structure of lignin. Pyrolysis (including fast-pyrolysis and fast-hydropyrolysis), hydrothermal processing, and liquefaction also process biomass as a whole, and while they only partially break down the biomass backbone, retaining structure as monomeric subunits of biomass components, they result in the formation of a large number of products, complicating separation procedures and further reactions. None of these options represent a selective way of processing lignin into discrete and usable products. Thus, lignin processing remains a significant challenge in the successful utilization of all components of biomass in a biorefinery.

### SUMMARY

Described herein is a process for conversion and upgrading of biomass to products. The process comprises converting the biomass to primary lignin-derivatives and primary cellulose/hemicellulose derivatives, catalytically treating the primary lignin-derivatives to produce secondary lignin-derived products, and treating the primary cellulose/hemicellulose-derivatives to produce secondary cellulose/hemicellulose-derived products.

### BRIEF DESCRIPTION OF THE FIGURES

FIG. 1 is a flow diagram depicting the disclosed process for production of fuels and chemicals from both the lignin and cellulose/hemicellulose components of biomass.

FIG. 2 depicts pathways for the production of renewable fuels (which are enclosed in a single-lined box) and chemicals (which are enclosed in a double-lined box) from the lignin portion of biomass.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In response to the need for an improved lignin processing method, disclosed herein is a new biorefinery concept for the production of renewable fine chemicals and liquid fuels that utilizes lignin based on recently reported discoveries that have overcome the challenge of processing lignin selectively into discrete products. Recent work, involving a single-step catalytic conversion of lignin into two methoxypropylphenol products from the lignin portion of a variety of whole biomass feedstocks followed by further catalytic hydrodeoxygenation of the methoxypropylphenols to hydrocarbons, has made it feasible to employ the lignin portion of biomass first, while simultaneously leaving behind an essentially intact solid carbohydrate fraction from cellulose that can be further processed via traditional biorefinery methods (fermentation and liquid phase upgrading) or alternative processes (fast-pyrolysis, gasification). The notion of converting the lignin first, while retaining the valuable aromatic structure, as opposed to last or not at all, effectively rewrites the idea of a typical biorefinery. This new biorefinery will allow for usage of all components of a lignocellulosic biomass feedstock (lignin, cellulose, and hemicellulose), which is critical to maximization of fuel and chemical yield per acre, and additionally enables unprecedented opportunities for integration of products from all components of biomass to generate new conversion pathways to fuels and chemicals. In addition, the disclosed process contributes to designing a biorefinery that potentially encompasses multiple synergistic processes (as opposed to only one processing method) designed to minimize destruction of the biomass by following the most effective pathways to map the natural structure of biomass to products.

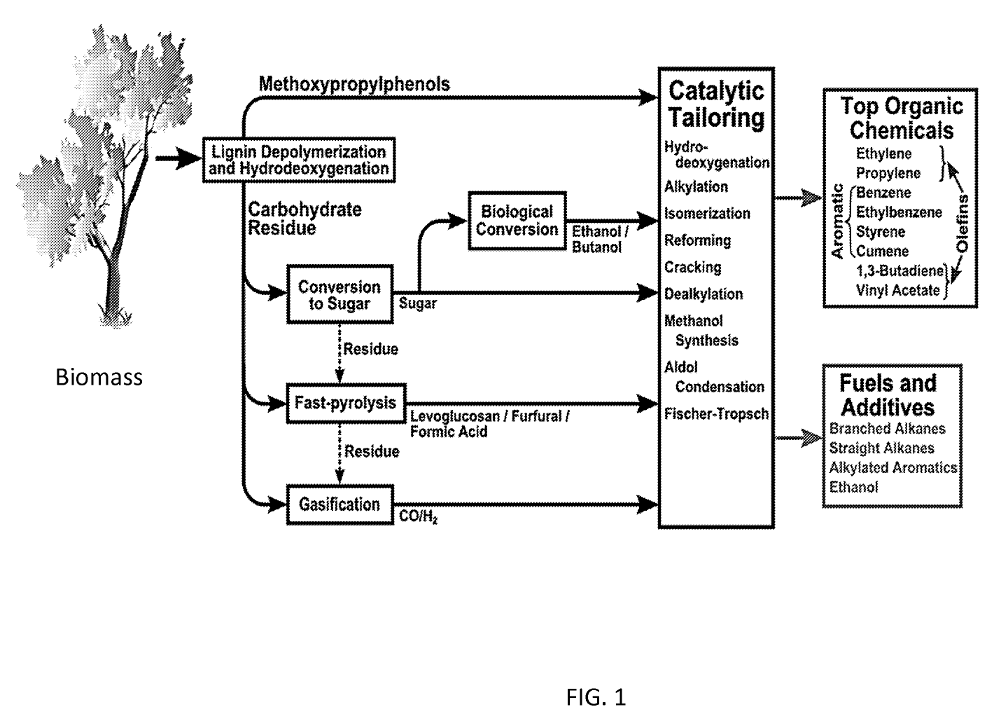

Referring to FIG. 1, a schematic of the disclosed process is presented. Still referring to FIG. 1, lignocellulosic biomass feedstock can be processed via the recently developed lignin depolymerization process that converts biomass into a) discrete methoxypropylphenol monomers derived from the lignin portion of biomass and b) a solid carbohydrate residue derived from the cellulose/hemicellulose portion of biomass. The methoxypropylphenol monomers can be used as-is for commodity chemicals, or tailored via a variety of catalytic processes to fuels (such as propylbenzene) or chemicals (such as BTX). The solid carbohydrate fraction can be further processed to fuels and chemicals by currently employed technologies such as biological conversion, catalytic conversion, or thermochemical processes such as fast-pyrolysis or gasification. The waste from certain processing steps, such as residues or char, could be utilized in the proposed synergistic biorefinery by feeding to a thermo-chemical unit (fast-pyrolysis or gasification) for conversion into a variety of intermediate products which can then undergo further catalytic processing. Additionally, the product mixture derived from traditional thermochemical processes (pyrolysis, gasification, or hydrothermal processing of biomass) could be separated into the lignin-derived and carbohydrate-derived fractions and upgraded via the corresponding biological or catalyst conversion processes. The biorefinery configuration would be established depending on the desired products, as well as other factors such as biomass feedstock, location, legislative and economic factors.

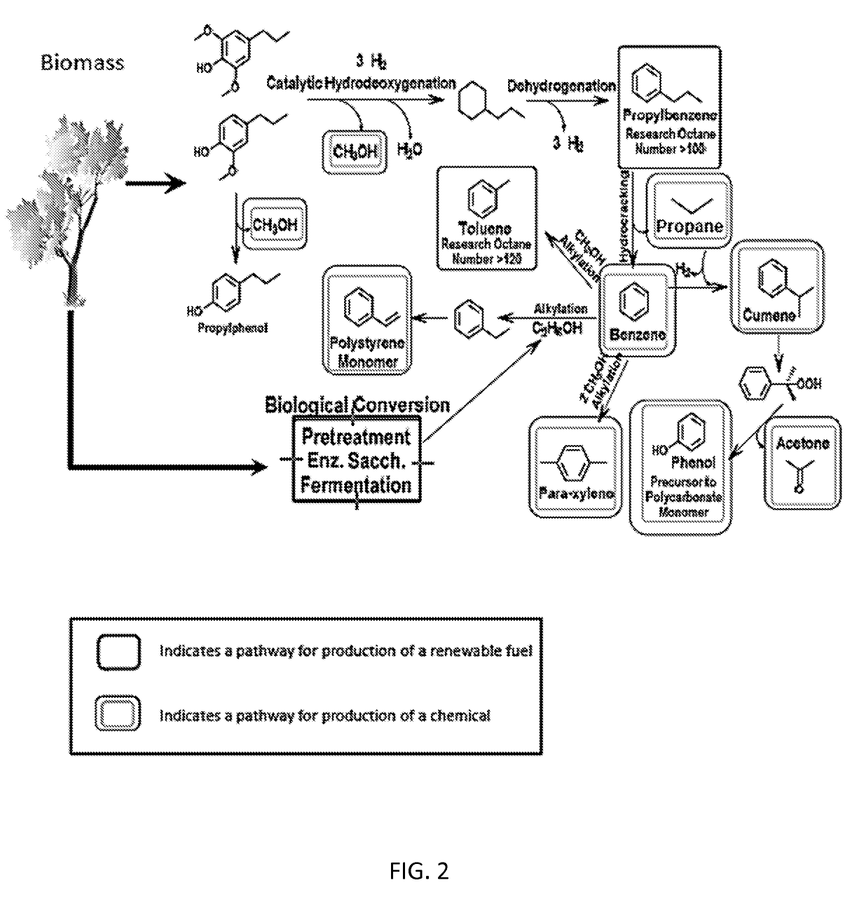

Now that a method for selective production of methoxypropylphenol monomers from lignin has been developed, many options that were previously not viable due to the complexity of the lignin product mixture can be envisioned for the conversion of these primary products into current fine chemical and energy commodities. As shown in FIG. 2, the lignin derived methoxypropylphenols can be used as-is for the fragrance industry (i.e. dihydroeugenol), or could be catalytically tailored via hydrodeoxygenation, dehydrogenation, hydrocracking, and de/alkylation reactions to a variety of fuels (such as propylbenzene, toluene, etc.) and chemicals (such as methanol, benzene, cumene, para-xylene, ethylbenzene, styrene, phenol, and acetone). Recently reported were: a) formation of propylphenol and methanol from the methoxypropylphenols and b) a greater than 97% yield of the hydrocarbon propylcyclohexane from the high-pressure, vapor-phase hydrodeoxygenation reaction of the methoxypropylphenols using a bimetallic catalyst. Propylcyclohexane can be converted via dehydrogenation to propylbenzene, which can be hydrocracked to benzene and propane. Benzene can be used as a chemical building block for production of fuels and chemicals via a variety of known conversion pathways, such as: alkylation of benzene with produced propylene (made from dehydrogenation of co-produced propane) to form cumene, alkylation with co-produced methanol to form toluene or xylenes, or alkylation with ethanol or ethylene to form ethylbenzene and styrene. The produced propylene could be sold for polymer production or could be converted further to acetone; which when reacted with cumene forms phenol, which could be further converted to bisphenol-A (a polycarbonate monomer).

Furthermore, options for lignin and carbohydrate chemical integration are possible, presenting new synergistic pathways for production of fuels and chemicals. For example, in FIG. 2, alkylation of benzene with two moles of methanol to form para-xylene is proposed, where the methanol can be sourced from cleavage of the methoxy groups from the starting methoxypropylphenols or can be produced from the breakdown of the carbohydrate fraction. Production of styrene is possible through an integrated pathway involving alkylation of benzene using ethylene or renewable ethanol produced from sugar fermentation. Alkylation of benzene with renewable methanol and ethanol are also possible routes for the production of high octane alkyl-aromatic fuels. Integration of both lignin and cellulose-derived compounds to produce additional products optimizes both carbon and conversion efficiency.

In addition to the pathways proposed above, which use a lignin-derived feedstock for the production of renewable benzene as a drop-in replacement to petroleum feedstocks, there are opportunities for the development of new, more-direct pathways to aromatic chemicals. Utilization of the natural aromatic lignin structure (aromatic ring, alkyl and oxygen substituents) enables a minimum number of conversion steps to generate renewable commodities, as opposed to the production of aromatics from petroleum which often occurs via a series of many steps. For example, the direct production of cumene can be accomplished from propylbenzene via isomerization of the propyl side group. Dealkylation of propylphenol can be a direct route to phenol and propylene. Propylbenzene can be directly hydrocracked to form styrene and methane.

By 2020, the NRC reports the U.S. will have 498 Mtons/year of sustainably available biomass (consisting of approximately 25% of lignin) which could potentially be used as a biorefinery feedstock. High yield production (>53% based on the reported 54% yield of methoxypropylphenols from lignin and 98% yield of propylcyclohexane from methoxypropylphenols) of propylcyclohexane from lignin enables production of renewable propane which can be dehydrogenated into propylene, the second highest by demand organic commodity chemical in the U.S. To put the impact of this into perspective, the produced methoxypropylphenols in accordance with the disclosure herein could produce ˜65.8 Mtons/year of propylcyclohexane. Of this total, 70% of the produced propylcyclohexane (˜46.1 Mtons/year) could meet the annual U.S. demand of ˜15.4 Mtons of propylene. In such a scenario, 30.7 Mtons/year of cyclohexane are co-produced which can be further converted to benzene, potentially exceeding the annual U.S. demand of ˜9.3 Mtons by ˜19 Mtons/year. The excess benzene is valuable as a high-octane fuel additive after alkylation to form 22.7 Mtons/year toluene, equivalent to 3.3% of the U.S. transportation demand (one possible alkylation agent is methanol). The remaining 19.7 Mtons/year (30%) of propylcyclohexane can be converted to ˜18.8 Mtons/year propylbenzene, equivalent to 2.8% of U.S. transportation demand, which is also attractive as a high-octane fuel additive. The residual lignin, cellulose, and hemicellulose in biomass still represent an attractive renewable feedstock for fuel production. Considering a standalone process, such as gasification/Fischer-Tropsch, the remaining biomass could supply ˜10.6% of U.S. transportation demand. However, the potential fuel production from the remaining biomass could be increased to ˜28.3% and 40.5%, respectively, of the U.S. transportation demand using integrated processes, such as the H₂Bioil and H₂CAR processes. This demonstrates the power of synergistic processes to improve conversion and energy efficiency.

Therefore, the disclosure presented herein represents a valuable addition to a novel biorefinery concept based on current advances in lignin processing which allows greater utilization of the various natural structures present in the lignocellulosic biomass feedstock. The use of the disclosed multiple processes to convert biomass feedstocks, based on the retention of the structure, will lead to synergistic benefits in their conversion into valuable products. The resulting new biorefinery employs all components of biomass, which enables additional pathways involving integration of components from the different fractions to provide new routes for the production of valuable renewable fuels and chemicals, specifically alkyl-aromatics using the aromatic substructure of lignin. It is estimated that by using the disclosed pathways, a new biorefinery would be able to meet the current U.S. demand for propylene and benzene chemicals with additional contribution of aromatics to the fuel market.

Additional disclosure is found in Appendix-A, filed herewith, entirety of which is incorporated herein by reference into the present disclosure.

While the disclosure has been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

### REFERENCES

1. Hayes, D. J., An examination of biorefining processes, catalysts and challenges. Catalysis Today, 2009. 145(1-2): p. 138-151.
2. Wellisch, M., et al., Biorefinery systems—potential contributors to sustainable innovation. 2010. Biofuels, Bioproducts and Biorefining. 4(3): p. 275-286.
3. Mandl, M. G., Status of green biorefining in Europe. Biofuels, Bioproducts and Biorefining. 2010. 4(3): p. 268-274.
4. Bozell, J. J., Connecting Biomass and Petroleum Processing with a Chemical Bridge. 2010. Science. 329(5991): p. 522-523.
5. J. E. Holladay, J. J. B., J. F. White, D. Johnson, Top Value-Added Chemicals from Biomass Volume II—Results of Screening for Potential Candidates from Biorefinery Lignin. 2007, Pacific Northwest National Laboratory (PNNL) and National Renewable Energy Laboratory (NREL).
6. Cherubini, F. and A. H. Strongman, Chemicals from lignocellulosic biomass: opportunities, perspectives, and potential of biorefinery systems. 2011. Biofuels, Bioproducts and Biorefining. 5(5): p. 548-561.
7. America's Energy Future: Technology and Transformation. 2009: The National Academies Press.
8. Agrawal, R. and N. R. Singh, Synergistic routes to liquid fuel for a petroleum-deprived future. 2009. p. 1898-1905.
9. Agrawal, R., et al., Sustainable fuel for the transportation sector. PNAS, 2007. 104(12): p. 4828-4833.

1. A process for conversion and upgrading of biomass to products comprising:
converting the biomass to primary lignin-derivatives and primary cellulose/hemicellulose derivatives;
catalytically treating the primary lignin-derivatives to produce secondary lignin-derived products; and
treating the primary cellulose/hemicellulose-derivatives to produce secondary cellulose/hemicellulose-derived products.

2. The process of claim 1, wherein at least one of the primary and/or secondary lignin-derived products and/or primary and/or secondary cellulose/hemicellulose-derived products is converted to form a tertiary product.

3. The process of claim 1, wherein the primary lignin-derivatives comprise methoxypropylphenol compounds.

4. The process of claim 1, wherein the primary lignin-derivatives comprise phenolic compounds.

5. The process of claim 1, wherein the primary cellulose/hemicellulose-derivatives comprise the fraction remaining after the primary lignin derivatives have been extracted from the biomass.

6. The process of claim 1, wherein the primary cellulose/hemicellulose-derivatives comprise sugar compounds.

7. The process of claim 1, wherein the primary cellulose/hemicellulose-derivatives comprise alcohols.

8. The process of claim 1, wherein the primary lignin-derivatives are upgraded to secondary lignin-derived products via a thermochemical process, comprising at least one of pyrolysis, fast-pyrolysis, fast-hydropyrolysis, liquefaction, catalytic processing, catalytic liquid-phase processing, catalytic vapor-phase processing, and hydrothermal upgrading.

9. (canceled)

10. The process of claim 1, wherein the primary cellulose/hemicellulose-derivatives are upgraded to secondary cellulose/hemicellulose-derived products via thermochemical processes, comprising at least one of pyrolysis, fast-pyrolysis, fast-hydropyrolysis, liquefaction, catalytic processing, catalytic liquid-phase processing, catalytic vapor-phase processing, and hydrothermal upgrading.

11. (canceled)

12. The process of claim 1, wherein the primary cellulose/hemicellulose-derivatives are upgraded to secondary cellulose/hemicellulose-derived products via at least one of biological conversion, fermentation, enzymatic saccharification, and anaerobic digestion.

13. The process of claim 1, wherein the primary cellulose/hemicellulose-derivatives are upgraded to secondary cellulose/hemicellulose-derived products via at least one of aqueous-phase pretreatment, hot water pretreatment, chemical pretreatment, dilute acid pretreatment, or catalytic pretreatment.

14. The process of claim 1, wherein the secondary lignin-derived products comprise aromatic compounds.

15. The process of claim 14, wherein the aromatic compounds include comprise phenolic compounds.

16. (canceled)

17. (canceled)

18. The process of claim 14, wherein the aromatic compounds comprise methoxyphenol compounds.

19. (canceled)

20. (canceled)

21. (canceled)

22. (canceled)

23. (canceled)

24. (canceled)

25. The process of claim 1, wherein the secondary lignin-derived products comprise saturated cyclic compounds.

26. (canceled)

27. (canceled)

28. The process of claim 1, wherein the secondary lignin-derived products comprise alcohols.

29. (canceled)

30. (canceled)

31. The process of claim 1, wherein the secondary lignin-derived products comprise ketones.

32. (canceled)

33. The process of claim 1, wherein the secondary lignin-derived products comprise light gases such as CH₄ and CO.

34. The process of claim 1, wherein the secondary lignin-derived products comprise straight chain and ring hydrocarbons.

35. The process of claim 34, wherein the hydrocarbons are alkenes.

36.-94. (canceled)
